(19) United States
(12) Patent Application Publication
(10) Pub. No.: US 2010/0166660 A1
(43) Pub. Date: Jul. 1, 2010

(54) METHODS OF MODELING MIGRAINE PAIN AND IDENTIFYING CANDIDATE COMPOUNDS FOR THE TREATMENT OF MIGRAINE

(76) Inventors: Frank Porreca, Tucson, AZ (US); Todd W. Vanderah, Tucson, AZ (US)

Correspondence Address: CLARK & ELBING LLP, 101 FEDERAL STREET, BOSTON, MA, 02110, US

(21) Appl. No.: 12/532,381

(22) PCT Filed: Mar. 21, 2008
(86) PCT No.: PCT/US08/03746

§ 371 Date: Mar. 8, 2010

Related U.S. Application Data
(60) Provisional application No. 60/896,839, filed on Mar. 23, 2007.

(51) Int. Cl. A61K 49/00; A61B 5/00; A61D 99/00

(52) U.S. Cl. 424/9.2; 600/557

(57) ABSTRACT

The present invention features animal models of migraine pain that can be used in a variety of ways, e.g., to identify compounds that reduce migraine pain or other migraine symptoms, to investigate behavioral changes correlated with the development and maintenance of a migraine-like state, and to better understand the mechanisms that underlie migraine pain.

### BACKGROUND OF THE INVENTION

In general, this invention relates to the fields of animal models for migraine pain and drug discovery.

Migraine headaches afflict millions of people each year, and as many as one in ten individuals is estimated to suffer from migraine pain at some point in their lives. Migraines are generally characterized by throbbing or pulsing pain on one side of the head, sometimes accompanied by sensitivity to light or sound, visual disturbances, or nausea.

Humans undergoing a migraine attack have been observed to develop an area of cutaneous allodynia (reduced threshold to pain such that initially non-painful stimuli become painful) that can expand over time. Initially, the region of hypersensitivity is restricted to the region of referred pain ipsilateral to the headache. During the course of the migraine, however, the area of allodynia can spread from this localized region to include large regions of the head and face, and eventually in some patients can also encompass areas outside the head and face.

While laboratory observations have shed some light on the sequence of events that may occur during a migraine attack, the mechanisms responsible for initiation of migraine headaches have remained obscure. Therefore, there is a need to develop animal models of migraine pain in order to be able to investigate more effectively the behavioral, physiological, and biochemical underpinnings of the development and maintenance of migraine pain.

### SUMMARY OF THE INVENTION

The invention features methods of modeling migraine pain in animals. These methods can be used, for example, to identify compounds that reduce migraine pain or other migraine symptoms. The models can also be used to advance our understanding of the biological mechanisms that underlie migraine pain.

Accordingly, the invention features a method of identifying a model for migraine pain in a test animal, including the following steps: (a) administering a first stimulus to the central nervous system of the test animal; (b) measuring a physical response of the test animal to a second stimulus at one or more predetermined times following the administration of the first stimulus; and (c) comparing the physical response of the test animal to the second stimulus to a physical response of a control animal to the second stimulus at one or more predetermined times following administration of a control stimulus to the control animal, wherein an increased physical response of the test animal to the second stimulus, compared to the physical response of the control animal to the second stimulus, identifies a model for migraine pain.

The invention further features a method of modeling migraine pain in a test animal, including the following steps: (a) administering a first stimulus capable of inducing migraine pain to the central nervous system of the test animal; (b) measuring a physical response of the test animal to a second stimulus at one or more predetermined times following the administration of the first stimulus; and (c) comparing the physical response of the test animal to the second stimulus to a physical response of a control animal to the second stimulus at one or more predetermined times following administration of a control stimulus to the control animal.

In these methods, the control stimulus can be administered, e.g., to the central nervous system of the control animal. In some instances, the test animal exhibits a reduction in 50% paw withdrawal threshold or 50% facial response threshold in comparison to the control animal.

The invention further features a method of identifying a compound that reduces migraine pain, including the following steps: (a) administering a first stimulus to the central nervous system of a test animal; (b) administering a candidate compound to the test animal; (c) measuring a physical response of the test animal to a second stimulus at one or more predetermined times following the first stimulus; and (d) comparing the physical response of the test animal to the second stimulus to a physical response of a control animal not receiving the candidate compound prior to the second stimulus, wherein a decreased physical response of the test animal to the second stimulus, compared to the physical response of the control animal to the second stimulus is indicative of the therapeutic efficacy of the candidate compound for migraine pain. Steps (a) and (b) can be carried out in either order, or simultaneously. In some instances, the test animal exhibits an increase in 50% paw withdrawal threshold or 50% facial response threshold in comparison to the control animal. In one instance, the control animal exhibits an increase in tactile hyperesthesia in comparison to an animal that did not receive the first stimulus; furthermore, the test animal can exhibit a decrease in tactile hyperesthesia in comparison to the control animal. Desirably, the first stimulus is capable of inducing migraine pain.

The candidate compound can be administered, e.g., to the central nervous system of the test animal. The candidate compound can be administered locally or systemically via any available route of administration, e.g., directly into discrete areas or nuclei of the brain, e.g., the rostral ventromedial medulla (RVM) or a brain ventricle, or onto the dura mater. Other routes of administration useful in the methods of the invention include intracranial, intracerebroventricular, intracerebral, parenteral, intravenous, intra-arterial, subcutaneous, intramuscular, intraorbital, ophthalmic, intraventricular, intracapsular, intraspinal, intrathecal, intracisternal, intraperitoneal, intranasal, aerosol, topical, suppository, or oral administration.

In any of the methods of the invention, the first stimulus can include a chemical stimulus, e.g., an inflammatory composition. Inflammatory compositions can include, e.g., one, two, three, or all four compounds selected from the group consisting of histamine, bradykinin, prostaglandin E2, and serotonin. Inflammatory compositions can also, or alternatively, include one or more compounds selected from the group consisting of a histamine agonist, a bradykinin agonist, a prostaglandin E2 agonist, and a serotonin agonist. Other chemical stimuli that can be used in the methods of the invention include calcitonin gene-related peptide (CGRP), a CGRP agonist, a nitric oxide donor, e.g., triglyceral nitrate (TGN) or s-nitrosylglutathione (SNOG), a cytokine, a cytokine agonist, or synthetic interstitial fluid.

The first stimulus can include a mechanical stimulus, e.g., indenting the dura of the test animal, with or without a chemical stimulus. The first stimulus is administered, for example, to the dura of the test animal.

In any of the methods of the invention, prior to administration of a first stimulus, a craniotomy can be performed on the test animal and/or control animal. In some instances, prior to step (a) and subsequent to the craniotomy, the animal can be fitted with an intracerebroventricular cannula or an intracranial guide tube. The guide tube can be placed, e.g., within 5 millimeters of the dura of the test animal. Desirably, the guide tube does not penetrate into or through the dura. Furthermore, prior to step (a) and subsequent to the craniotomy, the test animal can be fitted with a double cannula, which, for example, can be placed within 5 millimeters of the rostral ventromedial medulla, e.g., in contact with the rostral ventromedial medulla. A candidate compound can be administered through the double cannula.

In any of the methods of the invention, the second stimulus can include a tactile stimulus. In some instances, an animal receiving a first stimulus, e.g., an inflammatory composition administered to the dura, exhibits an increase in tactile hyperesthesia in comparison to an animal not receiving the first stimulus. The tactile stimulus can include, e.g., probing the test animal or control animal with a calibrated von Frey filament. The von Frey filament can be applied, for example, to the plantar surface of the hindpaw of the test animal or control animal; in the case of hindpaw application, the physical response of the test animal or control animal can include, e.g., a sharp withdrawal of the hindpaw, which can be measured, e.g., by determining the 50% paw withdrawal thresholds for the test animal and control animal. Alternatively, the von Frey filament can be applied, for example, to the face of the test animal or control animal; in the case of facial application, the physical response of the test animal or control animal can include, e.g., a sharp withdrawal of the head, or an attempt to grasp or bite the filament, either of which can be measured, e.g., by determining the 50% facial response thresholds for the test animal and control animal.

In any of the methods of the invention, the test or control animal can be a mammal, e.g., a rodent such as a rat, mouse, or guinea pig, or a primate, e.g., a non-human primate, such as a monkey, a chimpanzee, or an orangutan. The test animal and control animal can be of the same or different species.

The invention further features a method of inducing a behavioral change in an animal by administering a first stimulus to a component of the central nervous system, e.g., the dura. The behavioral change can be measured by any method, e.g., by measuring changes in paw withdrawal or facial response thresholds following administration of a second stimulus.

The invention further features a method for determining the propensity of a candidate compound to induce hyperalgesia in a human by infusing the candidate compound into a non-human mammal, e.g., a rat, for a period of at least 2 hours; applying a pain stimulus, e.g., a von Frey filament, to the non-human mammal; and determining the mammal's response to the stimulus, wherein the threshold of pain for the non-human mammal is indicative of the propensity of the candidate compound to induce hyperalgesia in a human. A lower pain threshold as compared to a control mammal not treated with a compound (such as treated with vehicle only) is indicative of the propensity for the candidate compound to induce hyperalgesia. A higher pain threshold as compared to a control mammal treated with vehicle or a triptan or other compound known to induce hyperalgesia in humans is indicative of reduced propensity, or lack thereof, for the candidate compound to induce hyperalgesia. In certain embodiments, the period may be at least 4, 6, 8, 10, 12, 14, 16, 18, 20, 22, or 24 hours. Preferably, the candidate compound is a compound that reduces migraine pain, e.g., as demonstrated using methods described herein.

By “administering a stimulus to the central nervous system” is meant introducing an agent or performing an action on a component of the central nervous system (CNS) of the subject being stimulated so as to induce a physiological or psychological activity or response of the component of the CNS, or of the subject as a whole.

By “candidate compound” is meant a chemical, be it naturally-occurring or artificially-derived, that is assayed for its ability to reduce migraine pain by employing one of the assay methods described herein. Candidate compounds can include, e.g., peptides, polypeptides, synthesized organic molecules, naturally-occurring organic molecules, nucleic acid molecules, and components thereof.

“Inflammatory composition” and “inflammatory soup” are used interchangeably herein and refer to a composition that is capable of causing inflammation, e.g., at the site of application.

“Inflammatory mediator cocktail” and “IM cocktail” are used interchangeaby herein and refer to an inflammatory composition that includes histamine, 5-HT (serotonin), bradykinin, and prostaglandin E2. In one instance, an IM cocktail includes 1 mM histamine, 1 mM serotonin, 1 mM bradykinin, and 1 mM prostaglandin E2.

By “physical response” is meant a physical action or motion induced by a stimulus. In rats, paw withdrawal and facial response are examples of physical responses resulting from a stimulus, e.g., a tactile stimulus. A physical response can be a single response to a stimulus or a statistical measure or other function of multiple responses (e.g., mean, median, mode, minimum, or maximum). Specifically included in the term “physical response” are the 50% paw withdrawal threshold and the 50% facial response threshold, as defined herein.

By “stimulus” is meant an agent or action that induces a physiological or psychological activity or response. For example, a chemical stimulus includes one or more chemicals that are capable of affecting an animal. A chemical stimulus can include an inflammatory composition. A mechanical stimulus includes any action involving physical contact with the animal that is capable of affecting the animal, e.g., applying pressure to a part of the animal. A tactile stimulus includes any stimulus that involves the sense of touch of the animal being stimulated, e.g., a mechanical stimulus of the skin. A control stimulus is a stimulus that induces a known response from the animal being stimulated. For example, a control stimulus can be a stimulus that causes a minimal effect and is used as a negative control for purposes of comparison to the effect caused by a test stimulus.

By “tactile hyperesthesia” is meant an increased or altered sensitivity to a tactile stimulus. Tactile hyperesthesia can occur, e.g., in response to migraine pain.

By “50% facial response threshold” is meant a measure of the force required to cause a test subject to move its head in response to a tactile stimulus, as determined by the non-parametric method of Dixon (Dixon, 1980; Chaplan et al., 1994) described herein.

By “50% paw withdrawal threshold” is meant a measure of the force required to cause a test subject to withdraw its paw in response to a tactile stimulus, as determined by the non-parametric method of Dixon (Dixon, Ann. Rev. Pharmacol. Toxicol. 20:441-462, 1980; Chaplan et al., J. Neurosci. Meth. 53:55-63, 1994) described herein.

The methods of the invention offer multiple advantages over previously-available models or other methods of investigation. For example, the present invention features the use of a behavioral readout, allowing for rapid screening of numbers sufficient for statistical analysis and evaluation in a short time. This result is typically not possible with other methods, e.g., an electrophysiological method requiring complex equipment and support, and in which anesthetics are required, which may mask the optimal effects.

In addition, in the methods of the present invention, test animals can be used on more than one occasion. The ability to reuse animals reduces costs of experiments and is consistent with reducing the total number of animals used in experiments expected in modern-day protocols. Repeated use of test animals is difficult to achieve when using other methods, e.g., those featuring electrophysiological studies.

Furthermore, the measurements recorded in the methods of the invention are closely related to clinical measurements of human subjects, e.g., in which allodynia in humans is evaluated using stimulatory filaments. Thus, the methods of the present invention are likely to have improved predictability of efficacy in humans, relative to other methods.

Notably, the methods of the invention result in substantially less FOS expression within the nucleus caudalis compared to other approaches, indicating a reduced sensitization within the trigeminal system caused by the intracranial surgery; the reduced FOS expression is indicative of a more accurate model that does not suffer from systematic distortions from lingering effects of the surgery.

In addition, the animal models of the invention are not class- or mechanism-dependent. Migraine is a complex disorder with multiple genetic, biochemical, and physiological links; many other models are mechanism-based and therefore are only able to demonstrate activity of the specific mechanism on which they are based, e.g., serotonin 1D agonism. However, mechanism-specific approaches render impossible the identification of new treatments that utilize a different mechanism. For example, there is not a direct causal link between nitric oxide synthase inhibitors and specific serotonin response. Consequently, multiple mechanism-specific screening models would have to be employed to achieve the same result. In contrast, the models of the present invention, for the first time, incorporate a direct behavioral change in response to pain in an awake, intact animal, presenting an effective method to evaluate candidate compounds for treatment of migraine as well as allowing for the investigation of the contribution of numerous mechanisms to the progression of the migraine attack. Efforts to discover or develop compounds or therapies effective in the treatment of migraine pain are much more likely to succeed if a behavioral readout of relevance to the pain disorder can be utilized, as in the present invention.

Other features and advantages will be apparent from the following description and the claims.

### BRIEF DESCRIPTION OF THE DRAWINGS

FIG. 1 is a schematic illustration of dural inflammatory stimulation applied to an area overlaying the left transverse sinus (TS). The superior sagittal sinus (SSS) is also shown.

FIG. 2 is a schematic illustration of dural inflammatory stimulation applied 1 mm lateral to the midline and 1 mm anterior to the coronal suture, as indicated by the arrow in the figure.

FIG. 3 is a graph showing that dural inflammation produces tactile hyperesthesia of the hindpaw. Inflammatory mediator (IM) cocktail was administered to one group of rats through intracranial guide tubes, while vehicle was administered to a second group. 50% paw withdrawal thresholds were then measured at a series of time points and plotted as shown.

FIG. 4 is a graph showing that administration of either IM cocktail or s-nitrosylglutathione (SNOG), a compound known to induce migraines in a clinical setting, causes tactile hyperesthesia of the hindpaw. Results are shown two hours after treatment.

FIG. 5A is a graph showing that dural inflammation produces facial tactile hyperesthesia. IM cocktail was administered to one group of rats through intracranial guide tubes, while vehicle was administered to a second group. 50% facial response thresholds were then measured at a series of time points and plotted as shown. FIG. 5B is a graph showing an expanded view of the graph of FIG. 5A.

FIG. 6A is a graph showing that systemic administration of sumatriptan attenuates facial tactile hyperesthesia induced by dural inflammation. IM cocktail was administered to two groups of rats through intracranial guide tubes. Thirty minutes later, sumatriptan was administered systemically to the first group, while vehicle was administered to the second group. 50% facial response thresholds were then measured at a series of time points and plotted as shown. FIG. 6B is a graph showing an expanded view of the graph of FIG. 6A.

FIG. 7A is a graph showing that administration of bupivacaine to the RVM attenuates facial tactile hyperesthesia induced by dural inflammation. Four groups of rats were treated: the first group received vehicle to the dura and saline to the RVM; the second group received vehicle to the dura and bupivacaine to the RVM; the third group received IM cocktail to the dura and saline to the RVM; and the fourth group received 1M cocktail to the dura and bupivacaine to the RVM. Saline or bupivacaine was administered thirty minutes after administration of vehicle or IM cocktail in each case. 50% facial response thresholds were then measured at a series of time points and plotted as shown. FIG. 7B is a graph showing an expanded view of the graph of FIG. 7A.

FIG. 8 is a graph showing that administration of sumatriptan attenuates tactile hyperesthesia of the hindpaw induced by dural inflammation. IM cocktail was administered to two groups of rats through intracranial guide tubes. Sumatriptan (0.6 mg/kg, s.c.) was administered 10 minutes prior to IM cocktail to the first group, while vehicle was administered to the second group. 50% paw withdrawal thresholds were then measured at a series of time points and plotted as shown.

FIG. 9A is a graph showing that administration of bupivacaine to the RVM attenuates tactile hyperesthesia of the hindpaw induced by dural inflammation. Four groups of rats were treated: the first group received vehicle to the dura and saline to the RVM; the second group received vehicle to the dura and bupivacaine to the RVM; the third group received IM cocktail to the dura and saline to the RVM; and the fourth group received IM cocktail to the dura and bupivacaine to the RVM. Saline or bupivacaine was administered thirty minutes after administration of vehicle or IM cocktail in each case. 50% paw withdrawal thresholds were then measured at a series of time points and plotted as shown. FIG. 9B is a graph showing the results of an experiment identical to the one shown in FIG. 9A with the exception that saline or bupivacaine was administered two hours after administration of vehicle or IM cocktail in each case.

FIG. 10 is a graph showing that administration of N-Monomethyl-L-Arginine (L-NMMA) or sumatriptan, both of which are known to disrupt migraine attacks in a clinical setting, attenuates tactile hyperesthesia of the hindpaw induced by dural inflammation. L-NMMA was administered intravenously at 10 mg/kg ten minutes prior to dural application of IM cocktail; sumatriptan succinate was administered subcutaneously at 1 mg/kg five minutes prior to dural application of IM cocktail. Results are shown two hours after dural inflammatory treatment.

FIG. 11 is a graph showing that the intracranial guide tube model of the present invention produces greater tactile hyperesthesia of the hindpaw than an alternative model. Rats in one group received IM cocktail through intracranial guide tubes to the dura, while rats in the second group received 4× synthetic interstitial fluid through a plastic chamber to a different region of the dura than the first group. 50% paw withdrawal thresholds were then measured at a series of time points and plotted as shown.

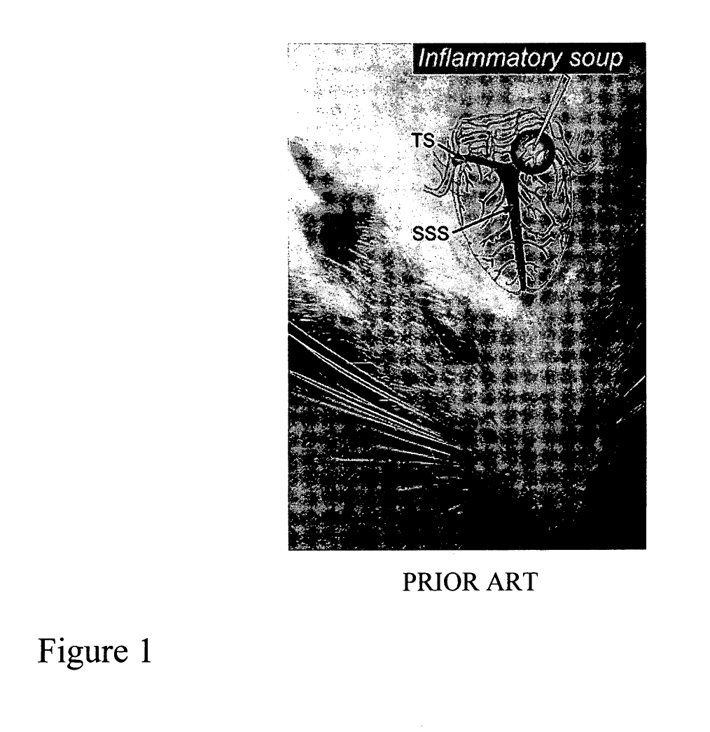

### DETAILED DESCRIPTION OF THE INVENTION

The present invention features animal models of migraine pain that can be used in a variety of ways, e.g., to identify compounds that reduce migraine pain or other migraine symptoms, to investigate behavioral changes correlated with the development and maintenance of a migraine-like state, and to better understand the mechanisms that underlie migraine pain.

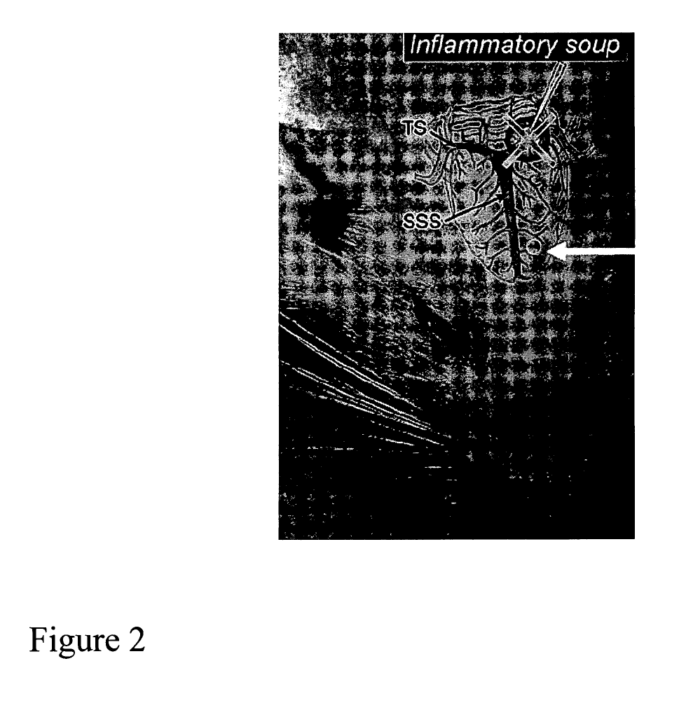

In one set of experiments, we have explored the development of a method of applying inflammatory soup to the dura in which a small intracranial guide tube is mounted within the left frontal bone of the skull, 1 mm lateral to the midline and 1 mm anterior to the coronal suture (FIG. 2). We have verified that this procedure allows guide tube placement that does not bruise the brain or puncture the dura. Further, our studies show very little FOS expression within subnucleus caudalis (Vc) at day 2, and at later time points (days 4 and 10) following surgery, suggesting that the any surgery-induced sensitization within the trigeminal system will have dissipated within this time-frame. Application of dye confirms the localization and spread of the inflammatory soup to neurons of the dura. Additionally, we have confirmed that placement of a cannula directed at the rostral ventromedial medulla (RVM) does not produce significant FOS expression in Vc on either days 4 or 7 after the intracranial guide tube to the dura and the RVM cannula are in place, allowing for the addition of inflammatory soup to the dura in the absence or presence of drug administration into the RVM.

Our studies, as described herein, include the following findings: (a) application of inflammatory soup to the dura produces facial allodynia; (b) application of inflammatory soup to the dura produces hindpaw allodynia; (c) application of bupivacaine to the rostral ventromedial medulla reliably blocks the inflammatory soup-induced facial and hindpaw allodynia, indicating an important central locus of action; (d) administration of classical anti-migraine drugs such as sumatriptan block the development of the allodynic response to facial and hindpaw stimulation; and (e) administration of a clinically effective anti-migraine compound, N-Monomethyl-L-Arginine (L-NMMA), blocks the development of allodynia.

Our experiments show that administration of the inflammatory soup alone is enough to induce allodynia, as determined by measuring the degree of tactile hyperesthesia, and there is no necessity for additional mechanical stimulation of the dura to achieve the desired sensitivity. Moreover, the fact that the animals are conscious when tested for allodynia, rather than inferring an effect from changes in electrophysiological recordings of relevant neuronal pathways, allows a direct behavioral readout. This is the first migraine model in which a pain-related response in multiple areas of the body can be actively quantified. In addition, the use of a nitric oxide donor applied to the dura in place of the inflammatory soup is sufficient to induce the same hyperalgesic state. Nitric oxide donors are known to induce migraine in susceptible individuals and are now regularly employed in human studies to investigate migraine in controlled conditions.

Thus, this is the first behavioral model of centrally-mediated migraine pain-related behavior in rats in which known stimulators of migraine can induce the behavioral response, and known migraine treatments can block the development of allodynia. An advantage of this model is that it is effectively class-independent in mechanism of action. Therefore, the model can be used to investigate the role of compounds and mechanism from multiple areas and domains thought to be associated with the development and maintenance of a migraine attack.

Uses of Migraine Models of the Invention

The migraine pain models of the invention can be used in a variety of ways. In one instance, the invention can be used to identify compounds that reduce migraine pain or other migraine symptoms, or prevent such symptoms from occurring, e.g., by establishing an experimental model of migraine pain in which tactile hyperesthesia or another symptom of migraine pain is induced in a test animal, and testing candidate compounds for the ability to eliminate, attenuate, or prevent the tactile hyperesthesia or other symptom. The models of the invention are also useful in investigating behavioral, physiological, or biochemical changes that correlate with the development and maintenance of a migraine-like state, as well as investigating the relative importance of various central structures on the development and maintenance of a migraine-like state from a behavioral, physiological, or biochemical perspective. In addition, the models can be used to investigate the development of centrally-mediated pain from chemical stimulation of the dura or other components of the central nervous system associated with other headache or pain states.

Migraine Pain Stimulus

Any stimulus that induces or models migraine pain, or a symptom of migraine pain, can be used in the invention. The stimulus can be applied anywhere on or in the body. For example, the stimulus can be applied to a component of the central nervous system, e.g., all or any part of the dura mater, the arachnoid, the pia mater, the brain, or the spinal cord. Suitable stimuli include chemical stimuli, e.g., an inflammatory composition, an agonist of a compound that causes inflammation, a calcitonin gene-related peptide (CGRP), a CGRP agonist, a nitric oxide (NO) donor, e.g., triglyceral nitrate (TGN) or s-nitrosylglutathione (SNOG), and/or a cytokine. Any compound known to induce or model migraine pain in a clinical setting, e.g., an NO donor, is useful as a migraine pain stimulus in the invention. Suitable stimuli also include mechanical stimuli, e.g., administration of physical pressure. Chemical and/or mechanical stimulation of the dura is effective in inducing migraine pain.

Any method of administering a migraine pain stimulus can be used in the invention. An exemplary method involves performing a craniotomy on the animal receiving the stimulus and inserting an intracranial guide tube. In one instance, a guide tube can be mounted within the left frontal bone of the skull, 1 mm lateral to the midline and 1 mm anterior to the coronal suture (FIG. 2). Preferably, the animal is allowed to recover prior to administration of the migraine stimulus, e.g., for one day, two days, three days, four days, five days, six days, one week, two weeks, three weeks, or one month. Recovery allows for a more accurate measurement of the animal's response to the stimulus.

Inflammatory Compositions

Particularly useful migraine pain stimuli include inflammatory compositions, e.g., any composition capable of causing inflammation of the dura or other component of the central nervous system. An exemplary inflammatory composition includes any or all of the following components, or variants thereof: histamine, serotonin, bradykinin, and prostaglandin E2. In one instance, an inflammatory composition can include 1 mM histamine, 1 mM serotonin, 1 mM bradykinin, and 1 mM prostaglandin E2. CGRP, NO donors, e.g., TGN or SNOG, and/or cytokines can also be used in an inflammatory composition. In addition, agonists of any of these components, e.g., histamine agonists, serotonin agonists, bradykinin agonists, prostaglandin E2 agonists, CGRP agonists, or cytokine agonists, can additionally or alternatively be used. Suitable concentrations for any or all of these components can be, e.g., 1 μM, 10 μM, 100 μM, 1 mM, 10 mM, or 100 mM. Suitable volumes of administration can be, e.g., 100 nl, 1 μL, 10 μL, 100 μl, 1 ml, or 10 ml.

Control Stimulus

Control stimuli can be administered, e.g., in a similar manner to a test stimulus, without necessarily causing a similar response. For example, if a test stimulus includes administration of a given volume of active agent via a given route, a suitable control stimulus can include, e.g., administration of the same volume of non-active agent, e.g., the vehicle used to deliver the active agent, via the same route. Alternatively, a control stimulus can include administration of an agent known to have a particular activity. Control stimuli can be used, e.g., for purposes of comparison to test stimuli, and can be administered to the same or a different animal.

Second Stimulus

After administration of a migraine pain stimulus, a second stimulus can be administered, e.g., for the purpose of evaluating a migraine pain model or testing a candidate compound for the ability to reduce migraine pain. Any second stimulus that causes a detectable effect can be utilized in the invention. For example, tactile stimuli can be used, and can give rise to a physical response or otherwise detectable effect, e.g., movement. In one instance, von Frey filaments can be used, e.g., as described by Chaplan et al. (J. Neurosci. Meth. 53:55-63, 1994), to induce movement, e.g., paw withdrawal or facial response.

The second stimulus can be administered at any point concurrent with or subsequent to administration of a migraine pain stimulus. For example, second stimuli can be administered at one or more predetermined times after the migraine pain stimulus, e.g., after one minute, five minutes, ten minutes, twenty minutes, thirty minutes, one hour, two hours, three hours, four hours, five hours, six hours, twelve hours, twenty-four hours, two days, three days, or one week.

Physical Response

Any physical response induced by a stimulus and capable of being detected can be used in the methods of the invention. For example, common physical responses to tactile stimuli are, e.g., paw withdrawal or facial response. Physical responses can also include increased expression of the FOS oncogene product, e.g., in cells of the medullary dorsal horn.

In one instance, administration of a sufficiently strong tactile stimulus to the hindpaw of a test animal, e.g., a rat, can cause the test animal to withdraw its paw, e.g., in a sharp motion. Administration of a sufficiently strong tactile stimulus to the face of the test animal can cause it to move its face and/or attempt to grasp or bite the object used to administer the tactile stimulus, e.g., a von Frey filament.

Physical response can be measured or otherwise determined in a variety of ways. In one instance, physical response is determined simply by noting the presence or absence of a particular physical action or motion, e.g., paw withdrawal or facial response, induced by a given stimulus. In another instance, physical response is measured by administering a series of stimuli, noting the resulting responses, and calculating a statistical measure or other function of the multiple responses, e.g., mean, median, mode, minimum, or maximum. Other exemplary functions used to determine physical response as a function of multiple measurements are the 50% paw withdrawal threshold and the 50% facial response threshold, as described in more detail herein.

In a test animal in which tactile hyperesthesia has been induced, e.g., by a migraine pain stimulus, the test animal is more sensitive to tactile stimuli and therefore exhibits a physical response at a lower tactile threshold than would otherwise occur in the absence of the migraine pain stimulus. In this case, the animal exhibits an increased physical response, i.e., an increased tendency to respond to tactile stimuli, relative to a control animal not subjected to the migraine pain stimulus.

In contrast, in a test animal in which tactile hyperesthesia has been attenuated, e.g., by a compound capable of reducing migraine pain and administered after a migraine pain stimulus, the test animal is less sensitive to tactile stimuli and therefore exhibits a physical response at a higher tactile threshold than would otherwise occur in the absence of the compound capable of reducing migraine pain. In this case, the animal exhibits a decreased physical response, i.e., a decreased tendency to respond to tactile stimuli, relative to a control animal not subjected to the compound capable of reducing migraine pain.

Candidate Compounds

Candidate compounds useful for testing in the methods of the invention can be identified from libraries of natural, synthetic, or semi-synthetic extracts, or from chemical libraries, according to methods known in the art. Candidate compounds can be chosen and/or tested individually or in combination with other candidate compounds. In general, any candidate compound capable of being assayed for its ability to reduce migraine pain, e.g., by employing one of the assay methods described herein, is useful in the methods of the invention.

Administration of candidate compounds may begin before, during, or after administration of a migraine pain stimulus, e.g., at one or more predetermined times relative to administration of a migraine pain stimulus, e.g., one minute, five minutes, ten minutes, twenty minutes, thirty minutes, one hour, two hours, three hours, four hours, five hours, six hours, twelve hours, twenty-four hours, two days, three days, or one week before or after administration of a migraine pain stimulus, or simultaneously.

Compositions

Compositions utilized in the invention, e.g., candidate compounds or chemical stimuli, can be administered within a pharmaceutically acceptable diluent, carrier, or excipient, e.g., in unit dosage form. Conventional pharmaceutical practice can be employed to provide suitable formulations or compositions to administer to test animals. Methods well-known in the art for making formulations and compositions are found, for example, in Remington: The Science and Practice of Pharmacy, 20th ed., ed. A. R. Gennaro, 2000, Lippincott Williams & Wilkins, Philadelphia, and Encyclopedia of Pharmaceutical Technology, eds. J. Swarbrick and J. C. Boylan, 1988-1999, Marcel Dekker, New York.

Any appropriate route of administration may be employed, e.g., directly into discrete areas or nuclei of the brain, e.g., the rostral ventromedial medulla (RVM) or a brain ventricle, or onto the dura mater. Any intracranial administration can be performed via, e.g., an intracranial guide tube or an intracerebroventricular cannula. Other modes of administration useful in the methods of the invention include intracerebroventricular, intracerebral, parenteral, intravenous, intra-arterial, subcutaneous, intramuscular, intraorbital, ophthalmic, intraventricular, intracapsular, intraspinal, intrathecal, intracisternal, intraperitoneal, intranasal, aerosol, topical, suppository, or oral administration. Administration may be local or systemic.

Animals

Any animal may be used in the methods of the invention. For example, mammals, e.g., rodents such as rats, mice, or guinea pigs, can be used. Non-human primates, e.g., monkeys, chimpanzees, and orangutans, can also be used.

### EXAMPLES

The following examples are provided for the purpose of illustrating the invention and are not meant to limit the invention in any way.

### Example 1

### A Behavioral Model for Migraine

An experimental model of migraine pain was created by using neurogenic inflammation in order to induce whole-body allodynia in rats. As described below in further detail, rats were craniectomized and fitted with an intracranial guide tube to the dura. After recovering from the surgical procedure, animals that received an inflammatory dural stimulus via the intracranial guide tube were shown to exhibit tactile hyperesthesia in comparison to control animals, consistent with the symptoms of migraine pain.

Animals

Male Sprague Dawley rats (275-300 g) were purchased from Harlan Sprague Dawley (Indianapolis, Ind.). Animals were given free access to food and water. Animals were maintained on 12-hour light (7 am to 7 pm) and 12-hour dark (7 pm to 7 am) cycles. All procedures were in accordance with the policies and recommendations of the International Association for the Study of Pain and the National Institutes of Health guidelines and use of laboratory animals as well as approved by the Animal Care and Use Committee of the University of Arizona.

Migraine Cannulation

Male Sprague Dawley rats were anesthetized using ketamine/xylazine (80-100 mg/kg, intraperitoneal). The top of the head was shaved using a rodent clipper (Oster Golden A5 w/size 50 blade), and the shaved area was cleaned with betadine and 70% ethanol. Animals were placed into a stereotaxic apparatus (Stoelting Co., #51600) and the body core temperatures of 37° C. were maintained using a heating pad placed below the animals. Within the shaved and cleaned area on the head, a 2 cm incision was made using a scalpel with a #10 blade, and any bleeding was cleaned using sterile cotton swabs. Location of bregma and midline bone sutures were identified as references, and a small hole 1 mm in diameter was made using a hand drill without breaking the dura but deep enough to expose the dura. Two additional holes (1 mm in diameter)4 to 5 mm from the previous site were made in order to mount stainless steel screws (Small Parts, #A-MPX-080-3F) securing the intracranial guide tube through which an inflammatory soup could be delivered to induce experimental migraine. A modified intracerebroventricular (ICV) cannula (Plastics One, #C313G) was used as an intracranial guide tube and placed into the hole without penetrating into or through the dura. The ICV cannula was modified by cutting it to a length of 1 mm from the bottom of the plastic threads using a Dremel rotary tool and a file to remove any steel burrs. Once the intracranial guide tube was in place, dental acrylic was placed around the intracranial guide tube and stainless steel screws in order to assure that the guide tube was securely mounted. Once the dental acrylic was dry (i.e., after ten to fifteen minutes), the cap of the intracranial guide tube was secured on top to prevent contaminants from entering the intracranial guide tube, and the skin was sutured back using 3-0 silk suture. Animals were given an antibiotic injection (Amikacin C, 5 mg/kg, intramuscular), removed from the stereotaxic frame, and allowed to recover from anesthesia on a heated pad. Animals were placed in a clean separate rat cage for a 5 day recovery period.

Migraine Intracranial Guide Tube Injections

An injection cannula (Plastics One, C3131, cut to fit the modified intracranial guide tube) connected to a 25 μl Hamilton Syringe (Hamilton Co., #1702SN) by Tygon® tubing (Cole-Palmer, #95601-14) was used to inject 10 μl of an IM cocktail onto the dura.

Behavioral Testing

Prior to the day of migraine surgery, naïve rats were placed in suspended Plexiglas™ chambers (30 cm L×15 cm W×20 cm H) with a wire mesh bottom (1 cm²) and acclimated to the testing chambers for thirty minutes.

Paw withdrawal thresholds to tactile stimuli were determined in response to probing with calibrated von Frey filaments (Stoelting Co., #58011). The von Frey filaments were applied perpendicularly to the plantar surface of the hindpaw of the animal until it buckled slightly, and were held for three to six seconds. A positive response was indicated by a sharp withdrawal of the paw. The 50% paw withdrawal threshold was determined by the non-parametric method of Dixon (Dixon, Ann. Rev. Pharmacol. Toxicol. 20:441-462, 1980; also see, e.g., Chaplan et al., J. Neurosci. Meth. 53:55-63, 1994). An initial probe equivalent to 2.00 grams was applied, and if the response was negative, the stimulus was increased one increment; otherwise, a positive response resulted in a decrease of one increment. The stimulus was incrementally increased until a positive response was obtained, then decreased until a negative result was observed. This “up-down” method was repeated until three changes in behavior were determined. The pattern of positive and negative responses was tabulated.

The 50% paw withdrawal threshold was determined as (10^{[Xf+kδ}])/10,000, where Xf is the value of the last von Frey filament employed, k is the Dixon value for the positive/negative pattern, and δ is the mean (log) difference between stimuli. Only naïve animals with baselines of 11 to 15 grams were used in the experiment. Fifteen grams was used as the maximal cut-off. Five days post-migraine surgery, paw withdrawal thresholds were re-tested using the same habituation and von Frey procedure as stated above. Data were converted to percent “antiallodynia” by the following formula: percent antiallodynia=100×(test value−post-treatment baseline value)/(pretreatment baseline value−post-treatment baseline value). Only animals that demonstrated no difference in their tactile hypersensitivity as compared to their pre-migraine surgery values were used in all studies.

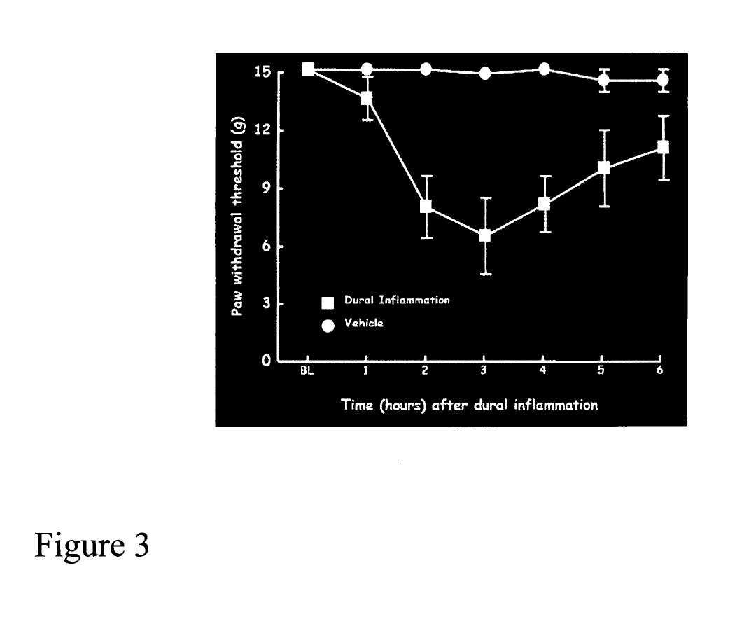

After establishing baseline paw withdrawal thresholds, individual animals were removed from the testing chamber, the cap of the migraine intracranial guide tube was removed, and animals received an injection of either an IM cocktail (1 mM histamine, 1 mM serotonin, 1 mM bradykinin, and 1 mM prostaglandin E2) or vehicle (i.e., control) at 10 μl volume via the intracranial guide tube over a five- to ten-second period. The IM cocktail was made fresh on the day of each experiment. The cap of the intracranial guide tube was replaced, individual animals were placed back into their corresponding testing chamber, and paw withdrawal thresholds were measured at one-hour intervals over a six-hour time course. A decrease in paw withdrawal threshold was observed for rats receiving IM cocktails in comparison to control rats (FIG. 3). Data were converted to percent “antiallodynia” by the following formula: percent antiallodynia=100×(test value−post-treatment baseline value)/(pretreatment baseline value−post-treatment baseline value).

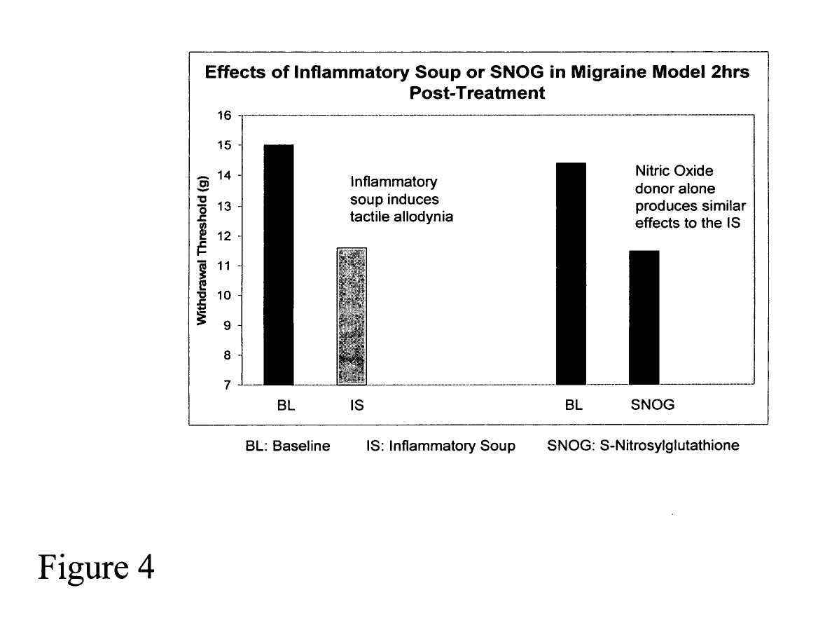

Further experiments demonstrated that the nitric oxide (NO) donor s-nitrosylglutathione (SNOG), when administered to the dura of test animals, produced a tactile hyperesthesia effect similar to that induced by administration of the IM cocktail (FIG. 4). SNOG belongs to a class of compounds that are known to induce migraines in the clinic, and therefore these experiments provided additional support for the correlation between migraine pain and tactile hyperesthesia observed in the model described herein.

In another set of experiments, facial response thresholds of the rats to tactile stimuli were determined. Calibrated von Frey filaments were applied perpendicularly to the midline of the forehead within a 3 mm diameter area just above the plane of the eyes, for six to eight seconds, until the filaments buckled slightly. A positive response was indicated by a sharp withdrawal of the head, which sometimes included an attempt to grasp and/or bite the filament. Special care was taken when applying the filaments to the forehead in order to prevent a positive facial response due to dynamic force and/or deflection of the hairs. The 50% facial response threshold was determined by the non-parametric method of Dixon. An initial probe equivalent to 1.00 gram was applied, and if the response was negative, the stimulus was increased one increment; otherwise, a positive response resulted in a decrease of one increment. The stimulus was incrementally increased until a positive response was obtained, then decreased until a negative result was observed. This “up-down” method was repeated until three changes in behavior were determined. The pattern of positive and negative responses was tabulated.

The 50% facial response threshold was determined as (10^{[Xf+kδ}])/10,000, where Xf is the value of the last von Frey filament employed, k is the Dixon value for the positive/negative pattern, and δ is the mean (log) difference between stimuli. Only naïve animals with baselines of eight grams were used in the experiment. Eight grams was used as the maximal cut-off.

Five days post-migraine surgery, the facial response thresholds were re-tested using the same habituation and von Frey procedure as stated above. Data were converted to percent “antiallodynia” by the following formula: percent antiallodynia=100×(test value−post-treatment baseline value)/(pretreatment baseline value−post-treatment baseline value). Only animals that demonstrated no difference in their tactile hypersensitivity as compared to their pre-migraine surgery values were used in all studies.

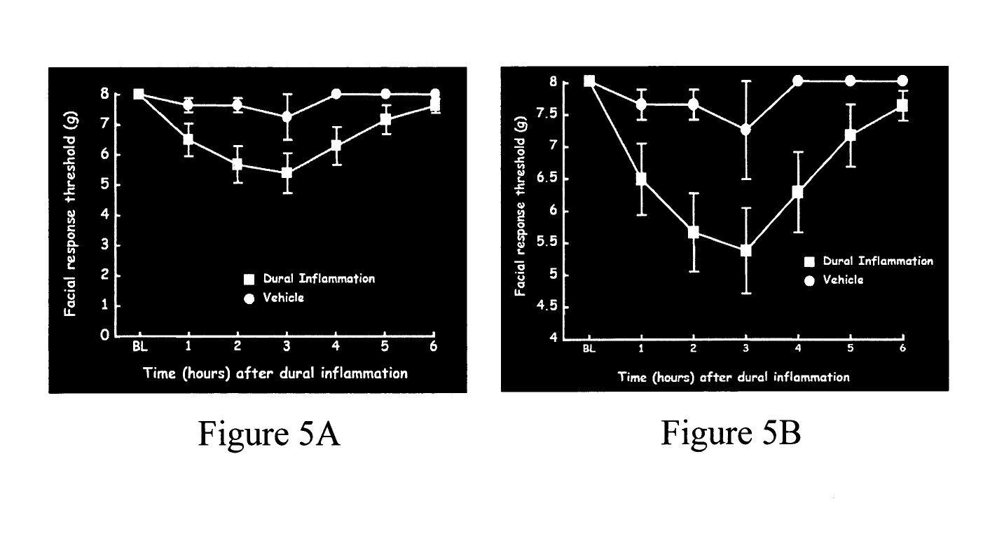

After establishing baseline facial response thresholds, individual animals were removed from the testing chamber, the cap of the intracranial guide tube was removed, and animals received an injection of either an IM cocktail (1 mM histamine, 1 mM serotonin, 1 mM bradykinin, and 1 mM prostaglandin E2) or vehicle at 10 μl volume via the intracranial guide tube over a five- to ten-second period. The IM cocktail was made fresh on the day of each experiment. The cap of the intracranial guide tube was replaced, individual animals were placed back into their corresponding testing chamber, and facial response thresholds were measured at one-hour intervals over a six-hour time course. A decrease in facial response threshold was observed for rats receiving 1M cocktails in comparison to control rats (FIGS. 5A-5B). Data were converted to percent “antiallodynia” by the following formula: percent antiallodynia=100×(test value−post-treatment baseline value)/(pretreatment baseline value−post-treatment baseline value).

### Example 2

### Testing Candidate Compounds for Anti-Migraine Activity

The experimental model for migraine pain described in Example 1 was used to test candidate compounds for activity in reducing migraine pain. As described below in further detail, the compounds sumatriptan (a known anti-migraine compound) and bupivacaine (a local anesthetic) were each found to reduce the tactile hyperesthesia observed in the experimental model in the absence of test compound.

Migraine & RVM Cannulation

Rats were anesthetized with ketamine/xylazine (80-100 mg/kg, intraperitoneal) for stereotaxic placement of bilateral cannulae in the RVM. The skull was exposed and a double cannula (26-gauge guide cannulae separated by 1.2 mm, Plastics One Inc., Roanoke, Va.) was directed toward the lateral portions of the RVM using co-ordinates from the atlas of Paxinos and Watson (1986) (anteroposterior, −11.0 mm from bregma, lateral, ±0.6 mm; dorsoventral, −7.5 mm from the dura mater). The guide cannula was cemented in place and secured to the skull by small stainless steel machine screws. The animals were allowed to recover for five days post-surgery before any pharmacological manipulations were made.

Modes of Administration of Candidate Compounds

RVM injections were performed using a previously implanted RVM double guide cannula (Plastics One, C235G-1.2). A 33-gauge injection cannula (Plastics One, C235I/Spc w/0.2 mm projection) connected to a 10 μl Hamilton Syringe (701RN) by Tygon® tubing (Cole-Palmer, 95601-14) was used to inject 1.0 μl (0.5 μl/side). At the termination of the experiments, pontamine blue was injected into the site of RVM injections and cannula placement was verified histologically. Data from animals with incorrectly-placed cannulae were not included within the data analysis. Data from animals with misplaced cannulae were included as off-site controls.

Subcutaneous injections were performed by manually holding the animal and inserting a 25-gauge disposable needle on a disposable 1 cc syringe into the abdominal region of the animal, assuring that the needle remained between the muscle and the skin of the animal. Injections of compounds were performed over a five-second period and were noted as positive by the development of an out-pocketing of the skin at the site of injection.

Oral delivery was accomplished by using an 18-gauge gavage needle attached to a 1 cc syringe.

Behavioral Testing

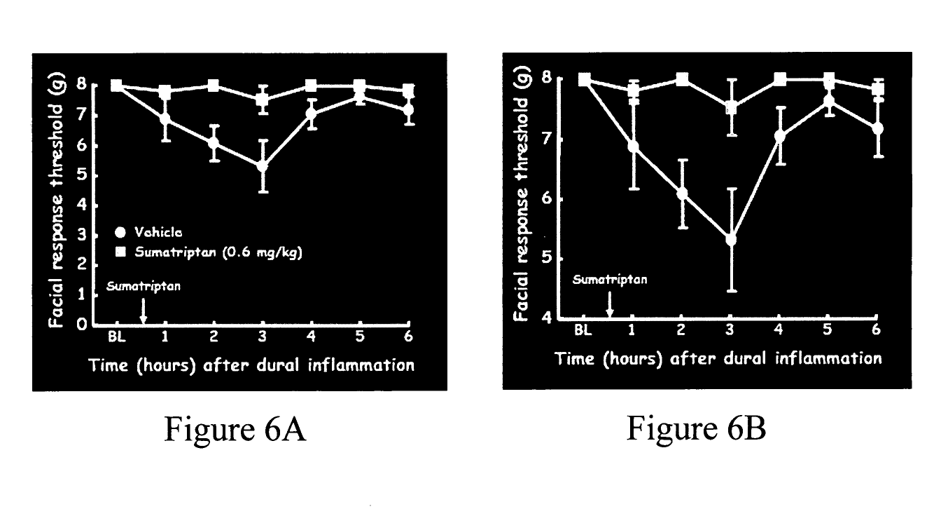

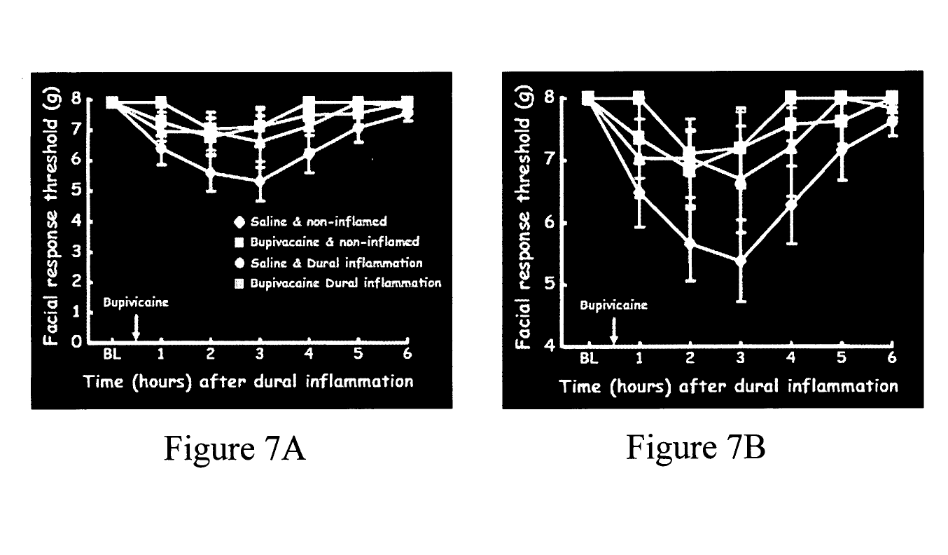

Behavioral testing was performed as described in Example 1, except that a candidate compound was administered to the test animals at a specified time following administration of the IM cocktail. Administration of systemic sumatriptan thirty minutes after administration of the IM cocktail caused an increase in facial response threshold in comparison to control rats that received a dural IM cocktail but no sumatriptan (FIGS. 6A-6B). Likewise, administration of bupivacaine in the RVM thirty minutes after administration of the IM cocktail caused an increase in facial response threshold in comparison to control rats that received a dural IM cocktail but no bupivacaine (FIGS. 7A-7B).

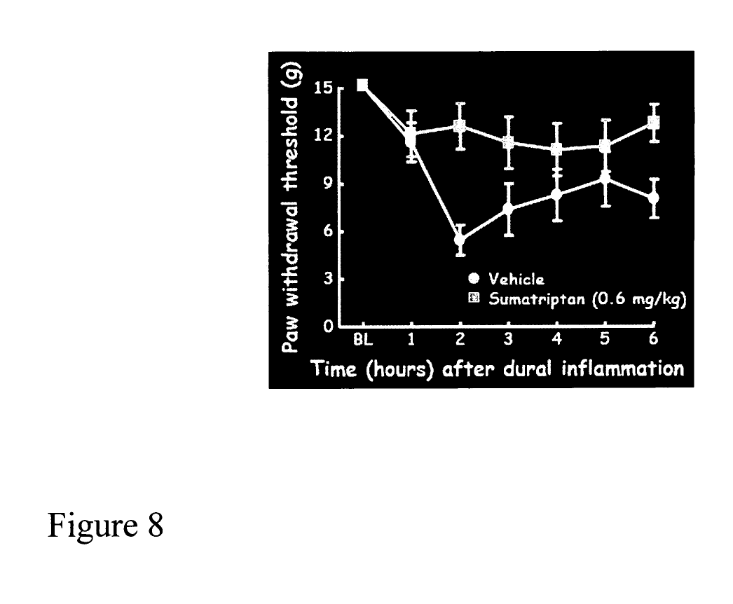

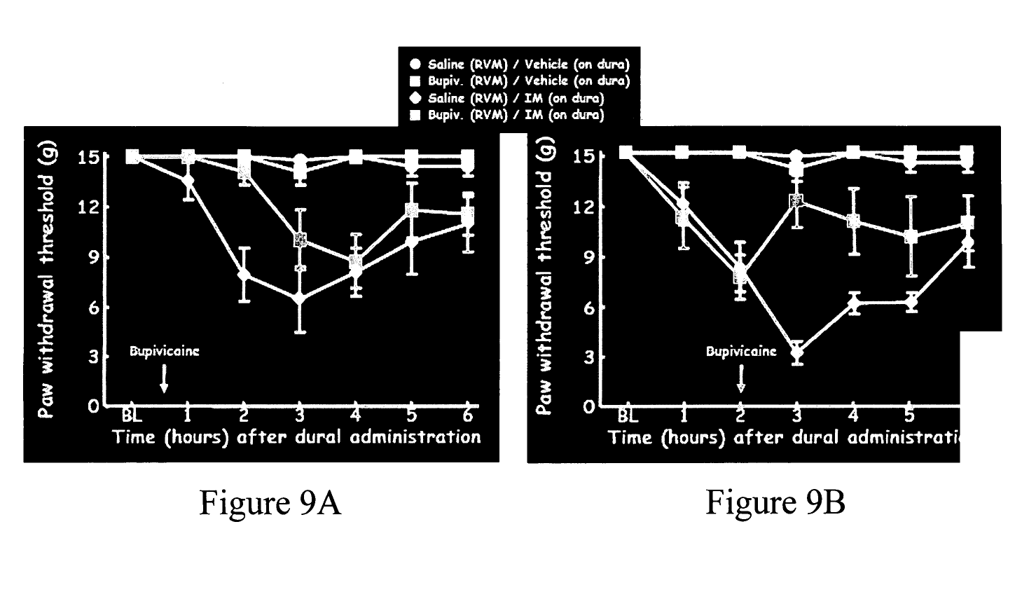

A similar set of experiments was conducted to assess the effect of candidate compounds on paw withdrawal threshold. Administration of sumatriptan ten minutes prior to administration of the IM cocktail caused an increase in paw withdrawal threshold in comparison to control rats that received a dural IM cocktail but no sumatriptan (FIG. 8). Likewise, administration of bupivacaine in the RVM thirty minutes after administration of the IM cocktail caused an increase in paw withdrawal threshold in comparison to control rats that received a dural IM cocktail but no bupivacaine (FIG. 9A). Test animals and control animals exhibited similar paw withdrawal thresholds until bupivacaine was administered two hours after administration of the IM cocktail, at which point a significant increase in test animal paw withdrawal threshold occurred in comparison to control animals (FIG. 9B).

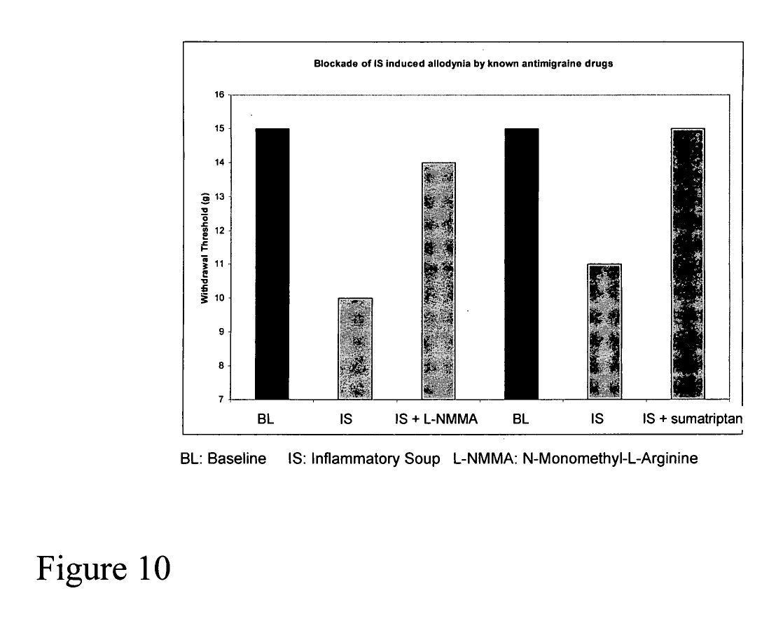

An additional set of experiments demonstrated that both sumitriptan and N-Monomethyl-L-Arginine (L-NMMA) prevent or ameliorate the development of tactile hyperesthesia in the migraine model of Example 1, as measured by determining paw withdrawal thresholds (FIG. 10). Both test compounds have been shown in the clinic to disrupt migraine attacks, and the compounds are from different chemical classes. Furthermore, these two compounds modify different molecular entities thought to be involved in migraine. Therefore, the models of the invention are effective across multiple mechanisms and represent a significant improvement over previous mechanism-based models.

### Example 3

### Comparison of Present Model to Alternative Model

Additional experiments were performed in order to compare the efficacy of the model of migraine pain described herein to an alternative model. Rats in the first experimental group were craniectomized and fitted with a modified intracerebro-ventricular (ICV) cannula, i.e., an intracranial guide tube, as described in Example 1. 10 μl of IM cocktail was administered through the intracranial guide tube to the dura of each rat in this group.

Rats in the second experimental group were craniectomized through the parietal bone, adjacent to the midline and above the transverse sinus, and a plastic chamber was affixed to the skull, as described in Malick et al. (Proc. Natl. Acad. Sci. U.S.A., 98:9930-9935, 2001). 20 μl of 4× synthetic interstitial fluid was administered through the plastic chamber to the dura of each rat in the second group.

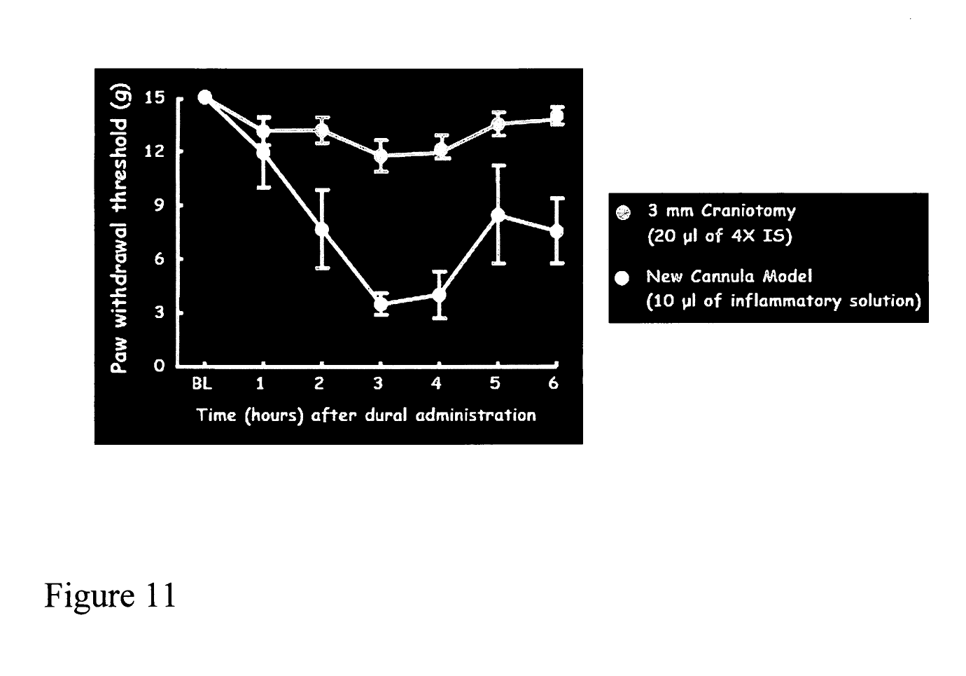

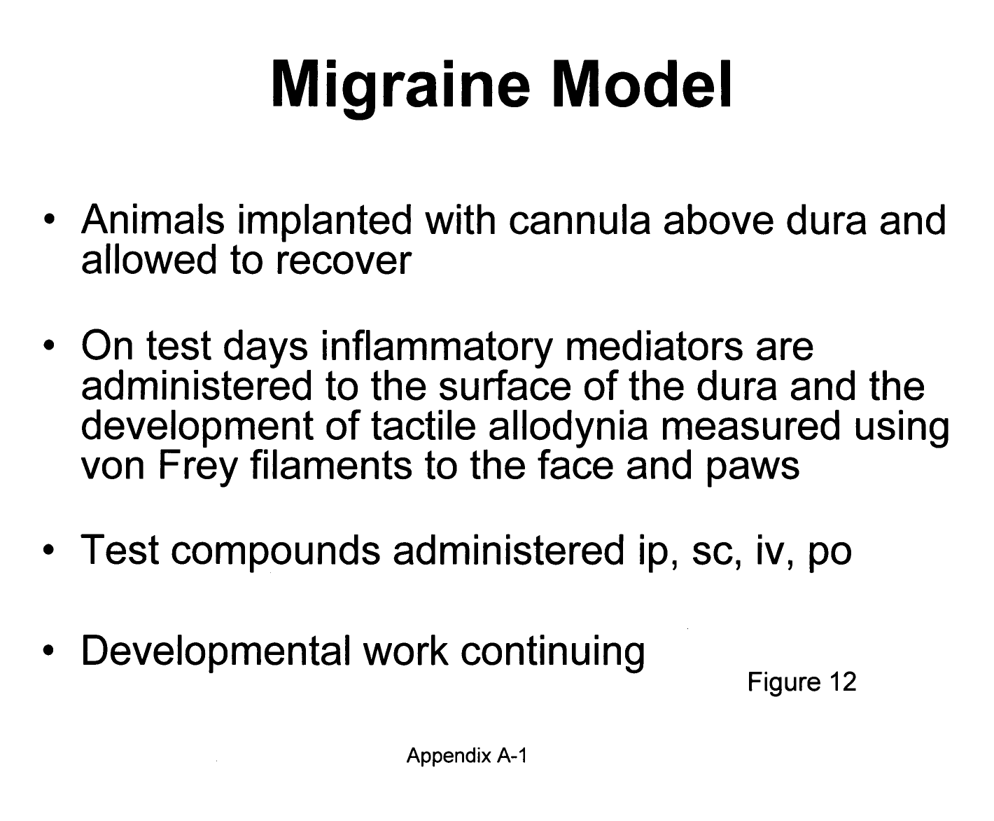

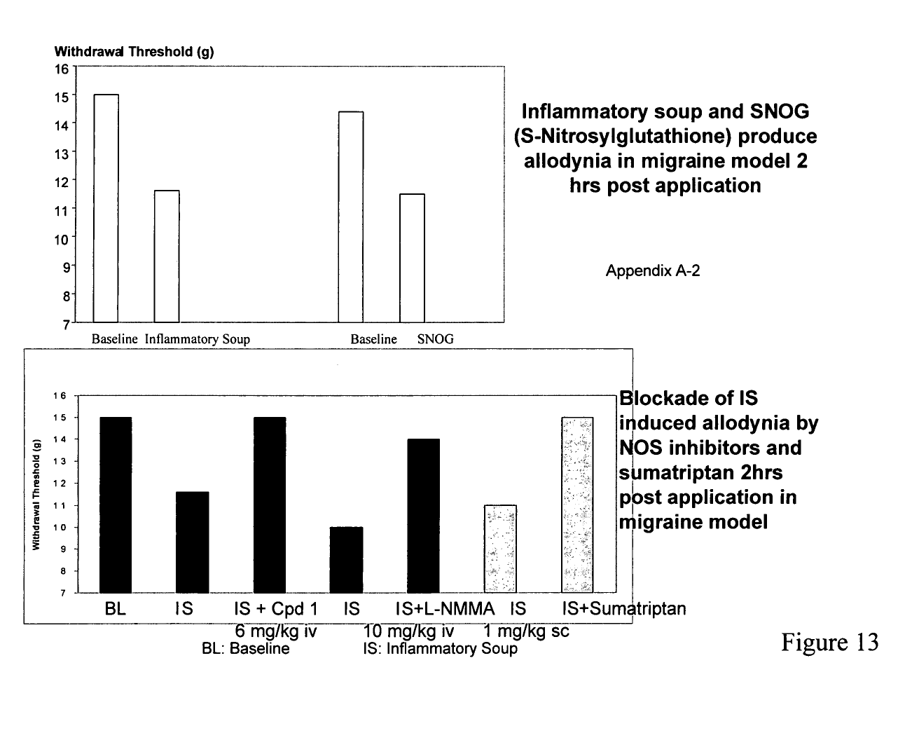

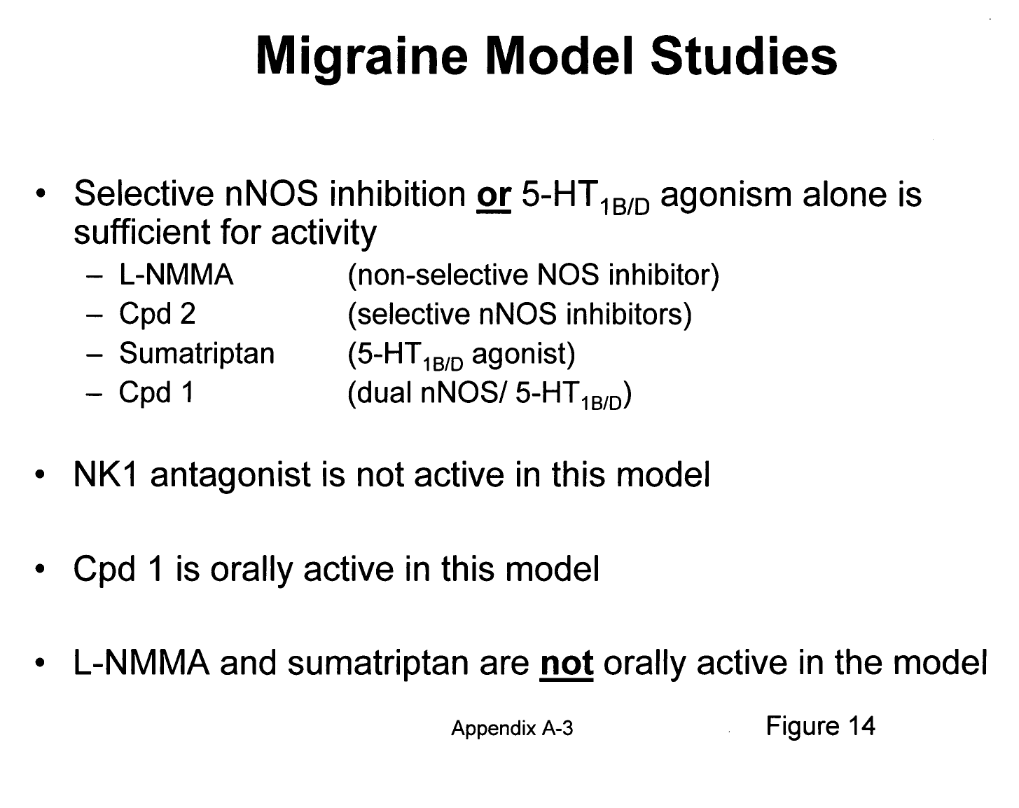

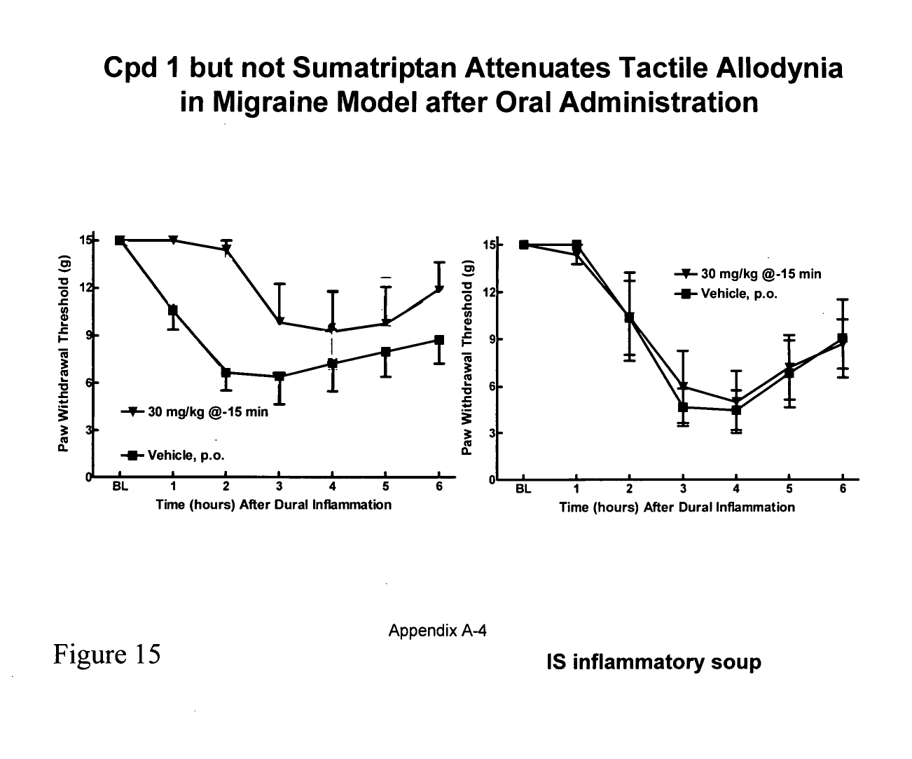

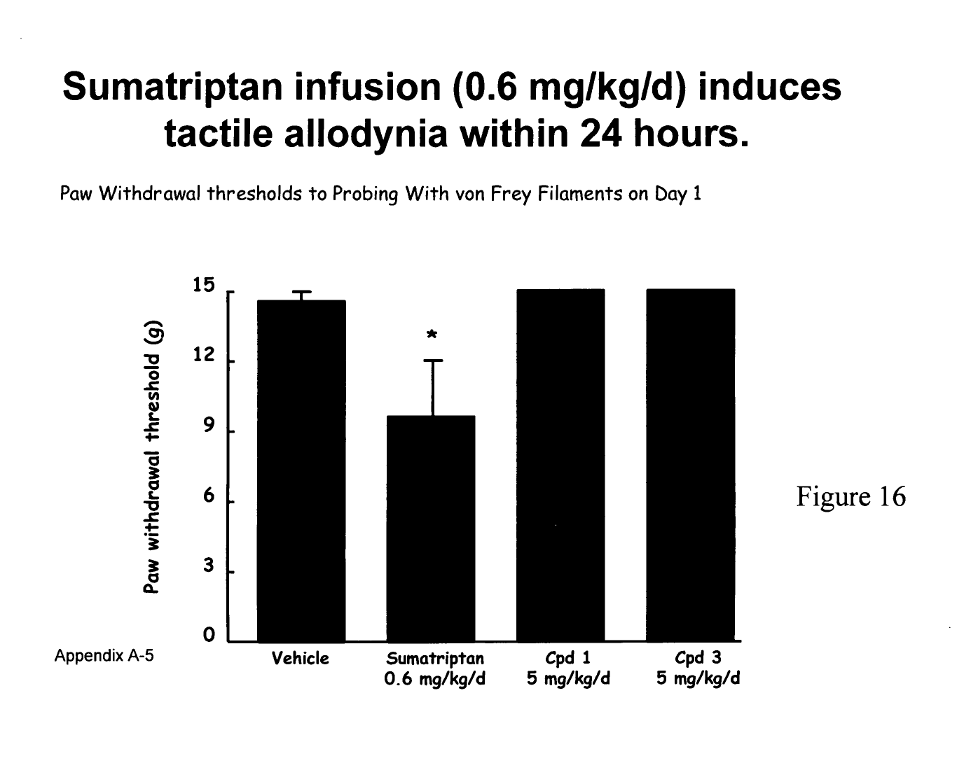

As in Example 1, the 50% paw withdrawal threshold for each experimental group was measured at one-hour intervals over a six-hour time course. As shown in FIG. 11, rats in the first group exhibited a reduced paw withdrawal threshold in comparison to rats in the second group. This result showed that the method employed with the first group produced a larger tactile hyperesthesia effect in comparison to the method employed with the second group, indicating that the first method produced an improved model of migraine pain.

### Example 4

With reference to Appendix A-3, cpd 1 has been shown to be orally active in migraine of the invention, while L-NMMA and sumatriptan were not. Furthermore, cpd 1, but not sumatriptan attenuated tactile allodynia in the migraine model of the invention after oral administration (Appendix A-4).

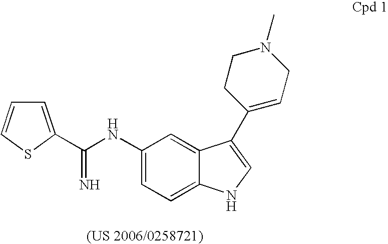

### Example 5

### Model for Allodynia

We have developed a model to determine the propensity for a compound to induce allodynia upon administration. Briefly, rats are infused continuously with a drug for a specified period of time. After the time, rats are probe with von Frey filaments to determine the paw withdrawal threshold. The compound may be administered for more than 2, 3, 4, 5, 6, 7, 8, 9, 10, 11, 12, 13, 14, 15, 16, 17, 18, 19, 20, 21, 22, 23, or 24 hours. Sumatriptan, a compound with a known propensity to induce hyperalgesia in humans, induces tactile allodynia after a 24 hour infusion at 0.6 mg/k/day. In contrast cpd 1 and another compound do not induce tactile allodynia after continuous infusion (Appendix A-5). This model indicates that cpd 1 is a candidate for a migraine treatment that will not induce hyperalgesia upon administration to a human, or induce it to a lesser extent than sumatriptan.

### Other Embodiments

All publications, patents, and patent applications mentioned in the above specification are hereby incorporated by reference. Various modifications and variations of the described method and system of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention.

Other embodiments are shown in Appendix A.

Other embodiments are in the claims.

What is claimed is:

1. A method for determining the propensity of a candidate compound to induce hyperalgesia in a human, said method comprising the steps of:
a) infusing said candidate compound into a non-human mammal for a period of at least 2 hours; and
b) applying a pain stimulus to said non-human mammal and determining said mammal's response,
wherein the threshold of pain for said non-human mammal is indicative of the propensity of said candidate compound to induce hyperalgesia in a human.

2. The method of claim 1, wherein said non-human mammal is a rat.

3. The method of claim 1, wherein said period is at least 4, 6, 8, 10, 12, 14, 16, 18, 20, 22, or 24 hours.

4. The method of claim 1, wherein said candidate compound is a compound that reduces migraine pain.

5. The method of claim 1, wherein said pain stimulus is a von Frey filament.
